(19) United States
(12) Patent Application Publication
(10) Pub. No.: US 2025/0281715 A1
(43) Pub. Date: Sep. 11, 2025

(54) SYSTEM AND METHOD FOR DIGITAL SCENT DELIVERY

(71) Applicant: OVR Tech, LLC, Burlington, VT (US)

(72) Inventors: Matthew Flego, Burlington, VT (US); Erik Cooper, Burlington, VT (US); Samuel Wisniewski, Burlington, VT (US); Sarah Socia, Burlington, VT (US); Gavin McCormick, Williston, VT (US); Lincoln C. Young, Freeville, NY (US); William B. Hathaway, Freeville, NY (US)

(73) Assignee: OVR Tech, LLC, Burlington, VT (US)

(21) Appl. No.: 19/070,129

(22) Filed: Mar. 4, 2025

Related U.S. Application Data
(60) Provisional application No. 63/561,836, filed on Mar. 6, 2024.

(51) Int. Cl. A61M 21/00; F03G 7/06

(52) U.S. Cl. CPC A61M 21/00; F03G 7/0614; A61M 2021/0016

(57) ABSTRACT

A system includes one or more vessels. Each of the vessels includes a scented medium between a first opening and a second opening. A first valve is disposed at the first opening of each of the one or more vessel. A second valve is disposed at the second opening of each of the one or more vessels. At least one of the first valve or the second valve of one of the one or more vessels is part of a first electromechanical actuation subsystem and another of the first valve or the second valve is a passive valve or is part of a second electromechanical actuation subsystem. A controller controls release of scented gas from at least one of the one or more vessels via the second opening to a corresponding outlet.

### RELATED APPLICATIONS

This application claims priority to and the benefit under 35 U.S.C. § 119(e) of U.S. provisional patent application No. 63/561,836, filed Mar. 6, 2024, entitled “SYSTEM AND METHOD FOR DIGITAL SCENT DELIVERY”, for which the entire contents are hereby incorporated by reference by their entirety herein.

### NOTICE OF MATERIAL SUBJECT TO COPYRIGHT PROTECTION

Portions of the material in this patent document are subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### SUMMARY

A system includes one or more vessels. Each of the vessels includes a scented medium between a first opening and a second opening. A first valve is disposed at the first opening of each of the one or more vessel. A second valve is disposed at the second opening of each of the one or more vessels. At least one of the first valve or the second valve of one of the one or more vessels is part of a first electromechanical actuation subsystem and another of the first valve or the second valve is a passive valve or is part of a second electromechanical actuation subsystem. A controller controls release of scented gas from at least one of the one or more vessels via the second opening to a corresponding outlet.

According to one aspect a system is provided. The system comprises a plurality of vessels, each of the plurality of vessels including a scented medium between a first opening and a second opening, a first valve disposed at the first opening of each of the plurality of vessels, a second valve disposed at the second opening of each of the plurality of vessels, wherein for each of the plurality of vessels, at least one of the first valve or the second valve is part of a first electromechanical actuation subsystem and another of the first valve or the second valve is a passive valve or is part of a second electromechanical actuation subsystem; one or more reservoirs holding a gas, one or more interconnections arranged to supply the gas from the one or more reservoirs to the first opening of the plurality of vessels, a controller configured to control release of scented gas from at least one of the plurality of vessels via the second opening of the at least one of the plurality of vessels to a corresponding outlet.

According to one embodiment, the scented gas results from the gas from one of the one or more reservoirs interacting with the scented medium in the at least one of the plurality of vessels. According to one embodiment, the scented medium is foam, wax, a paper substrate, gel, cotton ball, cotton pad, or porous polymer. According to one embodiment, the first electromechanical actuation subsystem and the second electromechanical actuation subsystem are a same type of electromechanical actuation subsystem. According to one embodiment, at least one of the first electromechanical actuation subsystem or the second electromechanical actuation subsystem includes a thermoelectric actuator. According to one embodiment, the thermoelectric actuator includes a shape memory alloy configured to move based on an application of a current and a stopper configured to cover at least one of the first opening or the second opening until movement of the shape memory alloy moves the stopper. According to one embodiment, the thermoelectric actuator includes Nitinol.

According to one embodiment, at least one of the first electromechanical actuation subsystem or the second electromechanical actuation subsystem operates based on an electrostatic or electrodynamic effect. According to one embodiment, at least one of the first electromechanical actuation subsystem or the second electromechanical actuation subsystem includes a piezoelectric actuator. According to one embodiment, the gas is pressurized air. According to one embodiment, the gas is liquified gas. According to one embodiment, the liquified gas is carbon dioxide. According to one embodiment, the system further comprising a fan or air pump coupled to the one or more reservoirs to urge flow of the gas from the one or more reservoirs. According to one embodiment, the controller is configured to communicate with an external device and control release of the scented gas based on communication from the external device. According to one embodiment, the controller is configured to communicate with the external device wirelessly or via a port. According to one embodiment, the system further comprising a cartridge configured to house the plurality of vessels. According to one embodiment, the system further comprising a cartridge interface, wherein the cartridge is configured to detach from the system and the cartridge interface is configured to couple to the cartridge when the cartridge is attached to the system.

According to one embodiment, the cartridge interface includes first openings configured to align, respectively, to the first openings of the plurality of vessels housed in the cartridge, and first gaskets configured to seal an interface between the first openings of the cartridge interface and the first openings of the plurality of vessels, respectively. According to one embodiment, the cartridge includes grooves defining each channel from the second opening of each of the plurality of vessels to each corresponding outlet and the cartridge interface includes channel gaskets configured to seal each of the channels. According to one embodiment, the cartridge includes a sheet covering a side of the cartridge that couples to the cartridge interface. According to one embodiment, each channel is between the sheet and one of the grooves. According to one embodiment, the sheet is a film or a laminate. According to one embodiment, the system further comprising a removable cover configured to cover the cartridge. According to one embodiment, the controller obtains information from the cartridge. According to one embodiment, the information identifies a scent stored in each of the plurality of vessels. According to one embodiment, the information is stored in an electrically erasable programmable read-only memory (EEPROM) of the cartridge. According to one embodiment, the information indicates allowed and disallowed combinations of scents. According to one embodiment, the information indicates usage of one or more of the plurality of vessels. According to one embodiment, the information is used to replace the cartridge. According to one embodiment, the information is used to refill or replace one or more of the plurality of vessels.

### BRIEF DESCRIPTION OF DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended as a definition of the limits of a particular example. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. In the figures:

FIG. 1A is a perspective view of aspects of a digital scent delivery device according to some embodiments;

FIG. 1B is a perspective view showing different aspects of the digital scent delivery device shown in FIG. 1A;

FIG. 2 is a perspective view showing a slidable cover of an exemplary digital scent delivery device according to some embodiments;

FIG. 3A is a perspective view of an exemplary digital scent delivery device according to some embodiments with an outer cover removed;

FIG. 3B shows the exemplary digital scent delivery device of FIG. 3A with a cartridge of vessels detached;

FIG. 4 is a perspective view of an exemplary digital scent delivery device according to some embodiments with the housing removed;

FIG. 5 shows the exemplary digital scent delivery device of FIG. 4 with a controller cover removed;

FIG. 6 is a cross-sectional view of a first side of an exemplary digital scent delivery device according to some embodiments;

FIG. 7 is a cross-sectional view of a second side of the exemplary scent delivery device shown in FIG. 6;

FIG. 8 is a block diagram showing an exemplary interface between a reservoir and a vessel 215 according to some embodiments;

FIG. 9 is a cross-sectional view of an exemplary digital scent delivery device according to some embodiments;

FIG. 10 is an expanded view of aspects of the cross-sectional view shown in FIG. 9;

FIG. 11 is an expanded view of aspects of the cross-sectional view shown in FIG. 9;

FIG. 12A is a block diagram illustrating a channel for scent delivery according to one embodiment;

FIG. 12B is a block diagram illustrating a channel for scent delivery according to one embodiment;

FIG. 12C is a block diagram illustrating a channel for scent delivery according to one embodiment;

FIG. 13 is a block diagram of an exemplary vessel of according to some embodiments;

FIG. 14A is a block diagram of a closed valve that is part of an actuation subsystem that uses thermoelectric effect according to exemplary embodiments;

FIG. 14B is a block diagram of a closed valve that is part of an actuation subsystem that uses thermoelectric effect according to exemplary embodiments;

FIG. 15A is a block diagram of the valve of FIG. 14A in an open position;

FIG. 15B is a block diagram of the valve of FIG. 14B in an open position;

FIG. 16 is a block diagram of a closed valve that is part of an actuation subsystem that uses piezoelectric effect according to exemplary embodiments;

FIG. 17 is a block diagram of the valve of FIG. 16 in an open position;

FIG. 18 is a block diagram of a closed valve that is part of an actuation subsystem that uses electrostatic effect according to exemplary embodiments;

FIG. 19 is a block diagram of the valve of FIG. 18 in an open position;

FIG. 20 is a block diagram of a digital scent delivery device according to some embodiments; and

FIG. 21 is a block diagram of a digital scent delivery device according to some embodiments.

### DETAILED DESCRIPTION

According to some implementations, a digital scent delivery device is provided that is capable of emitting scent to a user. The inventors appreciated the need for a commercially-available digital scent delivery device for controllably rendering scent in any number of applications. Scent may be added to digital experiences (e.g., games, art exhibits, shows) that may be audio, visual, or audio-visual. In some embodiments, the digital scent delivery device may be used directly with a device such as a mobile phone, gaming system or other device or system to render scent to a user. In other embodiments such as virtual reality (VR), augmented reality (AR), mixed reality (MR), or any of those environments (XR), scent may be released in coordination with other digital experiences. A variety of other applications may also benefit from the addition of a digitally actuated scent emitting device, which may be referred to as an olfactory device. For example, scent may be added to a two-dimensional media experience (e.g., television, movie, game) or an interactive environment (e.g., game, application). In the wellness space, a digital scent delivery device may be used for aromatherapy (e.g., to trigger scent to stimulate portions of the brain in a different way than audio or visual effects to address anxiety by promoting relaxation or to address other health issues) or in a training environment for enhanced realism.

According to some embodiments, a digital scent delivery device may be configured to render scent directly to a user. For example, the device may be placed close to a user (e.g., to a user's nose) and the amount of scent emitted may be limited such that a personal scent experience may be created without scent contamination among different users occupying the same space. Spatial features of the environment may be used to control which scent to emit and how much of the scent to emit. Scent delivery may be triggered by proximity, location, or activity, for example, in a three-dimensional environment (e.g., XR) or a two-dimensional representation. According to some embodiments, scent delivery may be based on spatial characteristics of odor impression in a game or an XR environment as described in U.S. Pat. No. 11,351,450. According to other embodiments, a digital scent delivery device may provide the same scent experience for a number of people. Passive and/or electromechanical actuation subsystems may be used to control emission of one or more scents while preventing leakage of other scents. In some applications, a scent delivery device may be attached, via brackets or another attachment mechanism, to another device (e.g., game component, VR component, any glasses or goggles, etc.) or may be worn (e.g., via a lanyard) or otherwise held near a person for scent delivery.

According to exemplary embodiments, an actuation subsystem may include a valve at a first (separable) portion of the digital scent delivery device and actuation elements at another (core) part of the device. A removable/replaceable cartridge may include vessels that hold the various scents. Each of the vessels may include a first opening for a medium (e.g., pressurized gas (e.g., air), liquified gas (e.g., carbon dioxide)) to enter the vessel and a second opening for scented medium to exit the vessel. This cartridge may be coupled to the digital scent delivery device, which includes a controller to control scent delivery. In some embodiments, a film or laminate on the cartridge may act as a valve on the first opening and/or second opening. The film or laminate may be attached to the cartridge via adhesive, ultrasonic or laser bonding, or heat sealing.

For explanatory purposes, a valve may be a component that contacts a medium and prevents or allows flow of the medium into or out of an opening, while actuation elements may be one or more components that affect the valve to control the flow of the medium into or out of the opening. In exemplary embodiments including the film or laminate acting as a valve on the first and/or second opening of the vessels of the cartridge, the actuation elements may be part of the digital scent delivery device to which the cartridge is coupled, as detailed. Because the valve (i.e., the component that contacts the medium) is part of the cartridge while the actuation elements, which do not contact the medium, are part of the core digital scent delivery device, the cartridge may be replaced with another cartridge with different scents without residual scent from the previously coupled cartridge contaminating the scents of the new cartridge.

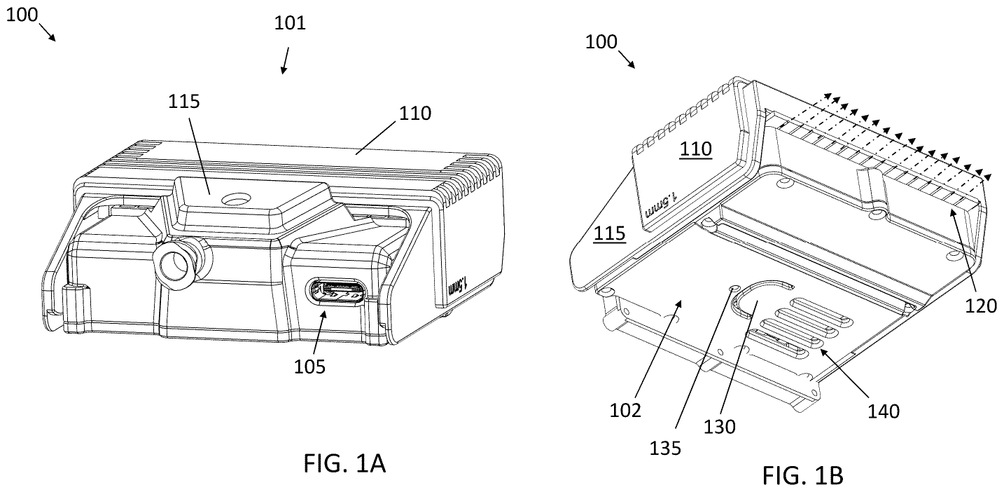

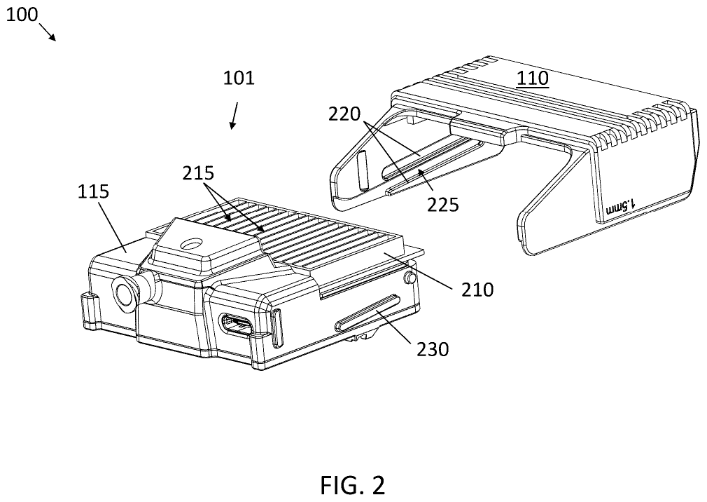

FIG. 1A is a perspective view of aspects of a digital scent delivery device 100 according to some embodiments. A first side 101 of the device 100 is visible in the view shown in FIG. 1A. A separable cover 110 is shown covering a portion of the first side 101 of the device 100. The cover 110 couples to a housing 115 of the device 100, as further discussed with reference to FIG. 2. The device 100 may communicate with any number and types of external devices to control emission of one or more scents. Non-limiting exemplary external devices that may communicate with and/or control scent delivery via the device 100 include an XR device, computer, cell phone, television, or gaming device. The external device may connect to the device 100 via a wire and connector to a port 105 (e.g., universal serial bus (USB) port) or may connect wirelessly (e.g., via Bluetooth or other protocol).

FIG. 1B is a perspective view showing different aspects of the digital scent delivery device 100 shown in FIG. 1A. A second side 102 of the device 100, opposite the first side 101, is visible in FIG. 1B. The outlets 120 available to emit scent are shown. Generally, one or a subset of the outlets 120 may emit scent at a given time. According to some embodiments, multiple scents may be emitted in turn or a blended scent may be provided through simultaneous actuation of two or more scent emissions. An on/off button 130 is shown on the second side 102 of the device 100. In alternate embodiments, any known power activation mechanism may be used. A light indicator 135 may emit light when the device 100 is turned on, for example. The light indicator 135 may emit different colors (e.g., green light when the device is on, red light when an error has occurred). Vents 140 for heat exhaust from aspects of the controller (e.g., processing circuitry and electronics, generally) are also shown on the second side 102 of the device 100.

According to various non-limiting embodiments, the device 100 may be held by, worn by, or attached to a person to controllably provide a personal scent experience. For example, the device 100 may be pinned, adhered, or otherwise attached to the clothing of a user. The device 100 may also be worn (e.g., on a necklace, bracelet, watch, ring, head wrap, goggles), embedded in or connected to other devices (e.g., headphones, computer, keyboard) or may be handheld or placed on a surface. The interaction or portability of the device 100 is not intended to be limited by any of the examples noted herein. According to some embodiments, the device 100 may be disposed near one or more people to provide a scent experience into the environment.

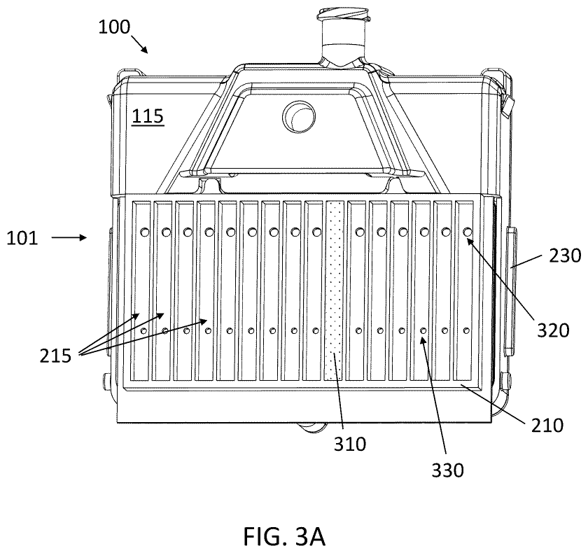

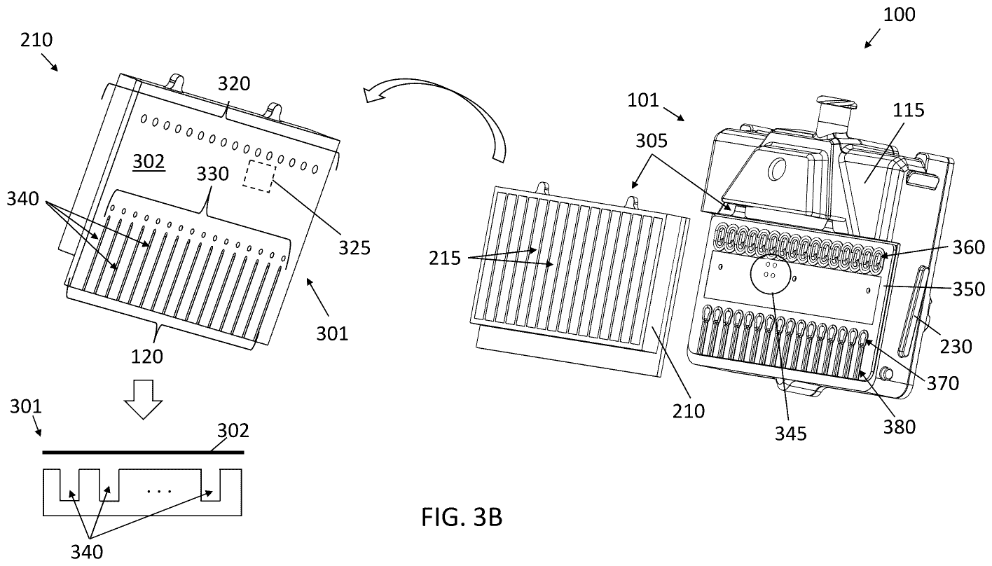

FIG. 2 is a perspective view showing a slidable cover 110 of an exemplary digital scent delivery device 100 according to some embodiments. The cover 110 is shown on the first side 101 of the device 100 and on the sides (perpendicular to the first side 101). As shown, the cover 110 may include a pair of sliders 220 on opposite sides of the cover 110 on an inner surface (i.e., a surface that couples to the device 100) with a gap 225 between each pair of sliders 220. The gap 225 between a pair of sliders 220 may fit a guide 230 on the device 100. Specifically, each side of the housing 115 of the device 100 may include a guide 230. The device 100 includes two guides 230 disposed on opposite sides of the housing 115. The guide 230 on each side of the device 100 may be inserted into or withdrawn from a corresponding gap 225 on each side of the cover 110. In the view shown in FIG. 2, the sliders 220 and gap 225 are visible on one side of the cover 110 and the guide 230 is visible on an opposite (rather than corresponding) side of the device 100. Although a slidable cover 110 is shown in discussed with reference to FIG. 2, any known mechanism may be used to moveably or removably attach the cover 110 according to alternate embodiments. With the cover 110 removed, a replaceable cartridge 210 is visible on the first side 101 of the device 100. The cartridge 210 may include a number of vessels 215 (generally one or more vessels 215), each holding a scent medium 310, as further discussed with reference to FIG. 3A.

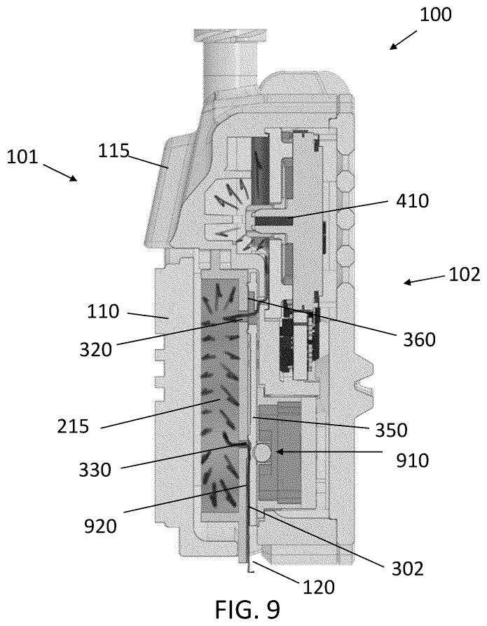

FIG. 3A is a perspective view of an exemplary digital scent delivery device 100 according to some embodiments with an outer cover 110 removed. As noted with reference to FIG. 2, a replaceable cartridge 210 is visible with the outer cover 110 removed. The cartridge 210 may include a number of vessels 215 holding different scent media 310. The vessels 215 are shown uncovered and most vessels 215 are shown as empty in FIG. 3A. Each vessel 215 has a first opening 320 and a second opening 330, as further discussed with reference to FIG. 9, that are visible for the empty vessels 215. An exemplary scent medium 310 is shown in one of the vessels 215 in FIG. 3A. According to different embodiments, two or more of the vessels 215 may include scent media 310. Non-limiting examples of scent media 310 include foam, wax, paper substrate, liquid, emulsion, gel, polyacrylamide, matrix, electrophoresis, Gellan Gum, cellulose material (e.g., cotton ball, cotton pad), porous polymer, and polysaccharide infused with a particular scent. A scent medium 310 may be molecules of the scent itself. The different scent media 310 may hold different scents for emission or, in alternate embodiments, two or more vessels 215 may include scent media 310 that emit the same scent.

FIG. 3B shows the exemplary digital scent delivery device 100 of FIG. 3A with the cartridge 210 detached from the device 100. The side 301 of the cartridge 210 that couples to the device 100 is also shown. Exemplary interconnects 305 on the cartridge 210 and device 100 attach the cartridge 210 to the device 100. In alternate embodiments, different numbers or types of interconnects 305 may be used. Generally, any known approach (e.g., snaping, sliding) to aligning and connecting the cartridge 210 to the device 100 may be used. Each cartridge 210 may be associated with an identifier or code that indicates the available scents and their associated vessels 215. For example, as indicated in FIG. 3B, the cartridge 210 may store identification and other information in an electrically erasable programmable read-only memory (EEPROM) 325 and the device 100 may include contact pins 345 to couple to the EEPROM 325 and read information from the EEPROM 325 of the cartridge 210. Approaches to storing and retrieving information are not limited to the EEPROM 325 and may involve, for example, a radio frequency identifier (RFID) tag or label affixed to the cartridge 210 or a machine readable code aligned with a reader of the device 100.

Detaching the cartridge 210 reveals a cartridge interlayer 350 that is the part of the device 100 that couples to the cartridge 210. The cartridge interlayer 350 includes gaskets 360, 370, 380 that correspond with first openings 320, second openings 330, and grooves 340 on a side 301 of the cartridge 210 that couples to the device 100. The gaskets 360, 370, 380 (e.g., silicon, rubber, other compliant material) form a seal around the openings 320, 330 and grooves 340 and may additionally act as a check valve, as further discussed with reference to FIG. 9.

The side 301 of the cartridge 210 that couples to the device 100 may be covered with a thin sheet 302 (e.g., film, lamination). The sheet 302 may be coupled to the cartridge 210 via adhesive, ultrasonic or laser bonding, or heat-sealing, for example. In an exemplary embodiment detailed with reference to FIGS. 9 and 11, the sheet 302 may act as a valve that allows or prevents flow out of the vessels 215 via the second opening 330 of each of the vessels 215. The sheet 302 may have punctures aligned with the first openings 320 of the vessels 215 such that the sheet 302 does not act as a valve for the first openings 320. In alternate embodiments, the sheet 302 may additionally or alternately act as a valve for each of the first openings 320.

FIG. 3B includes a view from the perspective of the outlets 120, looking into the cartridge 210. This view is shown with the sheet 302 separated from the cartridge 210 to more clearly show the grooves 340 in the body of the cartridge according to exemplary embodiments. When the cartridge 210 includes grooves 340, a groove 340 associated with a vessel 215 may define part of the channel 920 (FIG. 9) that facilitates flow of scented gas from the vessel to outside the device 100 (e.g., via an outlet 120). As discussed with reference to FIG. 11, for example, the sheet 302 above a particular groove 340 may pull away from the groove 340 based on pressure from exiting scented gas, for example. The sheet 302, groove 340, and gasket 380 that seals the sides of the sheet 320 define the channel 920 via which the scented gas flows out of the device 100.

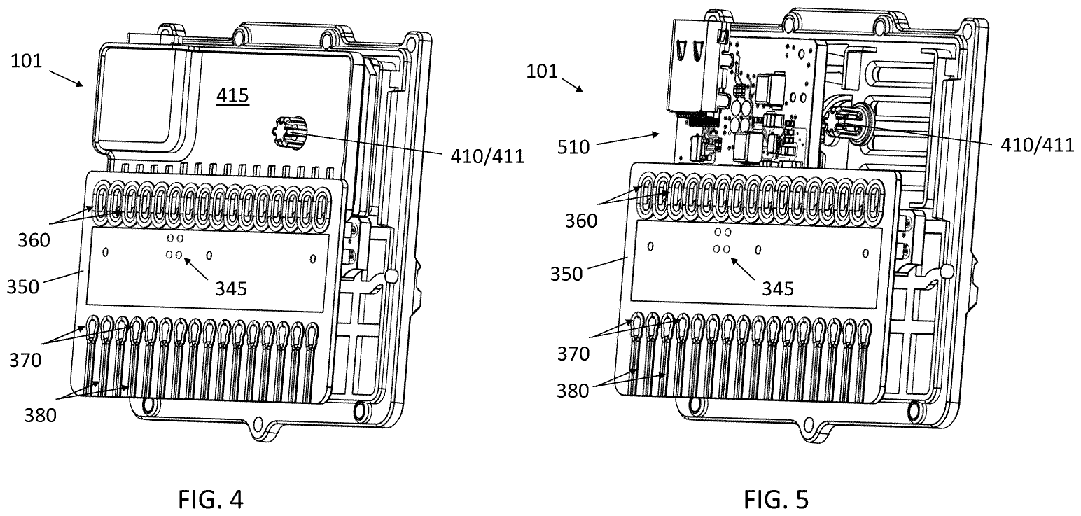

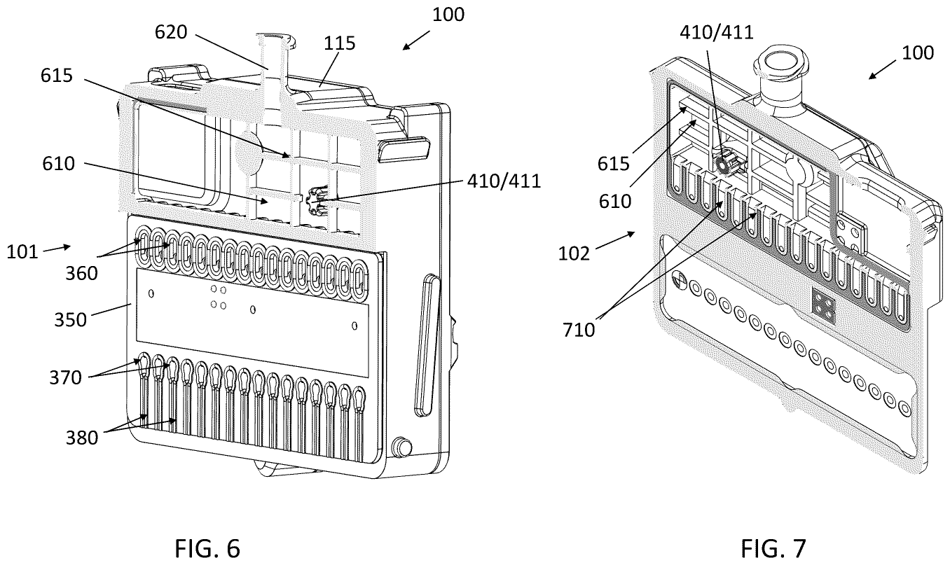

FIG. 4 is a perspective view of an exemplary digital scent delivery device 100 according to some embodiments with the housing 115 removed. A controller cover 415 is visible on the first side 101 of the device 100. A gas supply device 410 may be used to pressurize gas in a reservoir 610 (FIG. 6) or otherwise cause flow of the gas from the reservoir 610. As further discussed with reference to FIG. 6, the gas supply device 410 may be a pressure creator that pressurizes gas in the reservoir 610. In some embodiments, the gas supply device 410 may be a fan or pump used to actively direct gas out of the reservoir 610. According to different embodiments, the reservoir 610 may hold gas (e.g., air) or liquified gas (e.g., carbon dioxide). In the exemplary embodiment shown in FIG. 4, the gas supply device 410 is a piezoelectric-based device 411.

FIG. 5 shows the exemplary digital scent delivery device 100 of FIG. 4 with the controller cover 415 removed. The controller 510 generally refers to the various electronics and processing circuitry that may be included to communicate with an external device via wired or wireless communication, identify the cartridge 210 inserted into the device 100 (e.g., based on contact pins 345 contacting an EEPROM 325 of the cartridge 210), and control scent emission. Communication with the external device may include the controller 510 receiving a message comprised of a character string that indicates a desired scent. As noted with reference to FIG. 3B, the cartridge 210 that is coupled to the device 100 may include an EEPROM 325 that the controller 510 can read (e.g., via contact pins 345) to determine the scents and corresponding vessels 215 that are part of the cartridge 210. The EEPROM 215 may also store the amount of gas that has passed over each medium 310, the number of scent emission events for each medium 310, date of filling, and other identifying or use-indicating information. This usage information may be used to replace the cartridge 210, individual vessels 215, or their contents. That is, the vessels 215 may be removably placed in the cartridge 210 in addition to or alternately from the cartridge 210 being removably coupled to the device 100. Additionally or alternately, the vessels 215 may be refillable with scent and/or scented media 310.

The controller 510 may map a message string received from an external device to a particular scent (i.e., particular medium 310 in a particular vessel 215) and control the vessel 215 to emit the scent. The cartridge 210 may indicate (e.g., via information stored in the EEPROM) specific scent mixing formulas. That is, a message received by the controller 510 from an external device may include a string that corresponds with a mixed scent such that the controller 510 controls two or more corresponding vessels 215 to release the mixed scent. The cartridge 210 may additionally limit certain combinations of scent. For example, a manufacturer of a particular scent may specify that a new formulation that includes the scent may not be emitted. In that case, a message received from an external device that specifies the scent in combination with another scent may result in an error message (e.g., red light emitted by light indicator 135). For example, the controller 510 may issue an error when it receives a string that includes the identifier of the proprietary scent and the identifier of one or more additional scents. In this way, the rights to proprietary scents may be maintained when used in the device 100.

FIG. 6 is a cross-sectional view of a first side 101 of an exemplary digital scent delivery device 100 according to some embodiments. The cross-sectional view shows a reservoir 610 within the housing 115 that holds a gas. In the exemplary embodiment shown in FIG. 6, the gas is air obtained via the intake 620. In alternate embodiments, the gas may a gas other than air or may be a liquified gas. Structural features (e.g., bracing 615) may be included in the reservoir 610 to maintain structural integrity of the reservoir 610 and withstand pressure imposed by a pressure creator used as the gas supply device 410 according to some embodiments. In the exemplary case shown in FIG. 6, the outlet portion of a piezoelectric-based device 411 is shown within the reservoir 610. As previously noted, a fan or pump may be used, instead, to direct gas (e.g., air, liquified gas) out of the reservoir 610. The gas in the reservoir 610 may be directed into one or more vessels 215 via corresponding first openings 320. The scent that is emitted via one or more outlets 120 results from the gas from the reservoir 610 passing over the scent medium 310 within one or more vessels 215 prior to exiting the device 100 via associated one or more outlets 120, as further discussed with reference to FIG. 9.

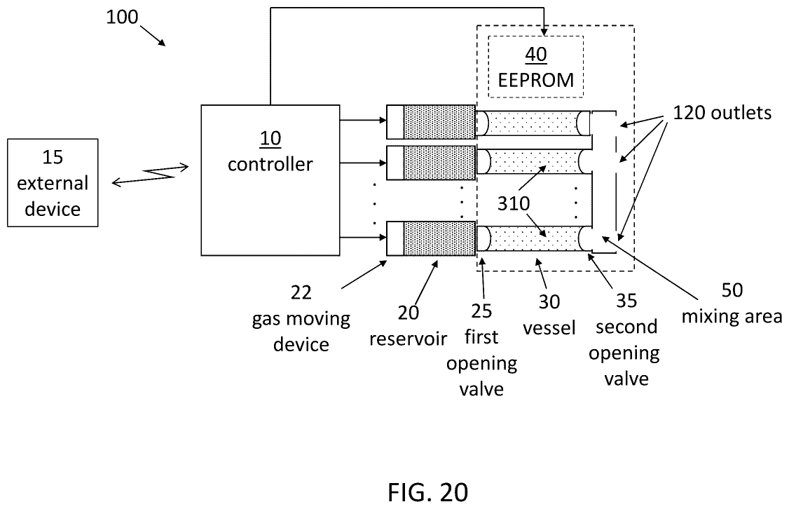

FIG. 7 is a cross-sectional view of a second side 102 of the exemplary scent delivery device 100 shown in FIG. 6. The reservoir 610 is shown with the bracing 615 and the gas supply device 410 is shown within the reservoir 610. As noted, the gas supply device 410 may be a piezoelectric-based device 411 that creates pressure within the reservoir 610 or a fan or pump that urges flow from the reservoir 610. Gas supply inlets 710 in the cartridge interlayer 350 to supply gas to corresponding vessels 215 of the cartridge 210 are visible in FIG. 7. While the single reservoir 610 provides gas for all of the gas supply inlets 710 according to the exemplary embodiment shown in FIG. 7, two or more reservoirs 610 may be used instead, as shown in FIG. 20. For example, each gas supply inlet 710 may be supplied by a separate reservoir 610.

As FIG. 6 indicates, the gas supply inlets 710 on one side of the cartridge interlayer 350 correspond with gaskets 360 on the opposite side of the cartridge interlayer 350. As indicated by FIG. 3B, the gaskets 360 are aligned with the first openings 320 in the vessels 215. The gaskets 360 may act as passive valves at the first openings 320 in the vessels 215. That is, initially, the passive valves (gaskets 360) may allow gas from the reservoir 610 to enter the vessels 215 via the first openings 320. When scent is emitted from a given vessel 215 (i.e., some of the gas allowed into the corresponding vessel 215 is emitted via a corresponding outlet 120), the gasket 360 corresponding with the given vessel 215 may allow additional gas to enter the vessel 215 via the first opening 320.

The gaskets 360 may prevent scented gas in the vessels 215 from flowing back through the first openings 320 and gaskets 360 to the reservoir 610. In this way, scent contamination (e.g., scent from one vessel 215 entering another vessel 215 via the reservoir 610) may be prevented. Because all the vessels 215 have gas in them at most times according to the embodiment including passive valves at the first openings 320, active electromechanical actuation subsystems (e.g., 910) may be used at the second openings 330 of the vessels 215 to prevent scents from all the vessels 215 being emitted via the outlets 120 at all times.

According to alternate embodiments, an active electromechanical actuation subsystem may be used at the first openings 320 of the vessels 215 instead. In this case, gas can be prevented from entering the vessels 215 and only a vessel 215 whose scent is to be emitted may have the valve at its first opening 320 controlled to permit entry of pressurized gas. As discussed with reference to FIG. 9, a passive valve may be used at the second openings 330 in this case. Generally, if a passive valve is used at the first openings 320 or the second openings 330, an active electromechanical actuation subsystem must be used at the other of the second openings or the first openings 320. Electromechanical actuation subsystems may be used at both the first openings 320 and the second openings 330.

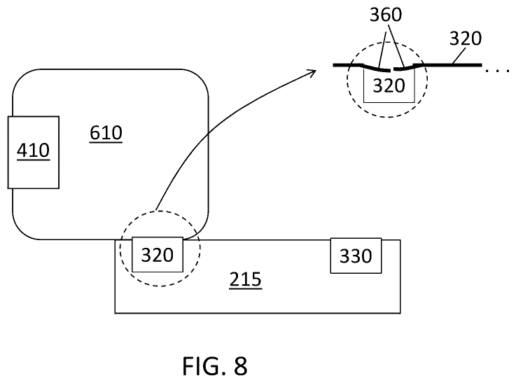

FIG. 8 is a block diagram showing an exemplary interface between a reservoir 610 and a vessel 215 according to some embodiments. According to the view in FIG. 8, additional vessels 215 may be behind the vessel 215 that is visible. The reservoir 610 shown in FIG. 8 may extend to supply the additional vessels 215 behind the visible vessel 215 or additional reservoirs 610 may be behind the reservoir 610 to supply one or a subset of the vessels 215 behind the visible vessel 215. The first opening 320 of the vessel 215 is shown at an interface with the reservoir 610. That is, one or more reservoirs 610 may directly interface with the vessels 215 rather than having the gas supply inlets 710 extend the volume of the reservoir(s) 610 to reach the vessels 215. As the expanded view illustrates, a sheet 320 (e.g., film, laminate) may be between the reservoir 610 and vessel 215 and have an opening at the first opening 320, and a gasket 360 may act as a passive valve between the reservoir 610 and vessel 215. Alternately, there may be an electromechanical actuation subsystem to control flow of gas (e.g., air, liquified gas) from the reservoir 610 into the vessel 215 and prevent backflow of scented gas from the vessel 215 into the reservoir 610. Alternate interfaces, as compared to the illustrated direct interface or the gas supply inlets 710 of FIG. 7, between the reservoir(s) 610 and vessels 215 may be used in alternate embodiments.

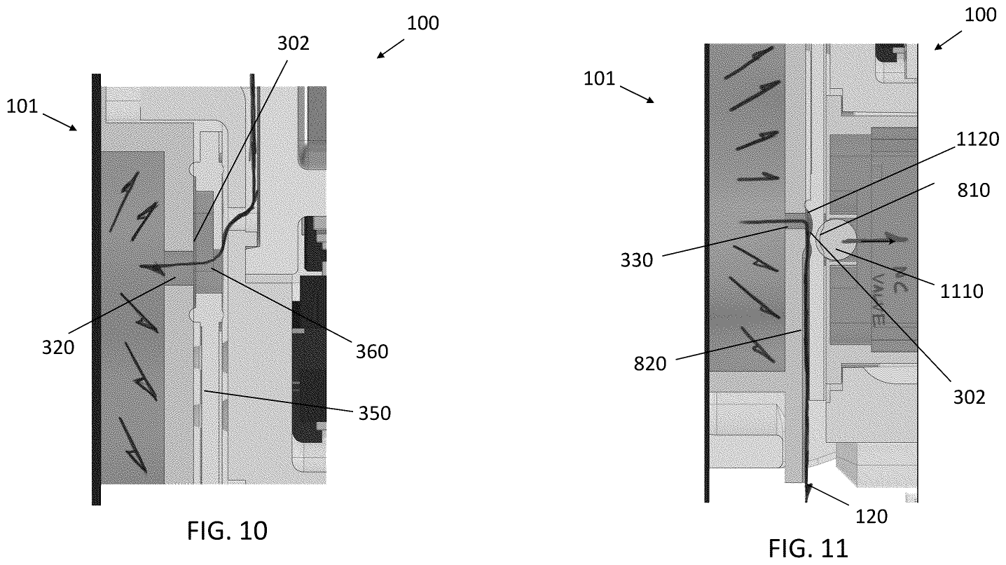

FIG. 9 is a cross-sectional view of an exemplary digital scent delivery device 100 according to some embodiments. The side cross-sectional view shown in FIG. 9 illustrates the flow associated with scent emission from one of the vessels 215. The first opening 320 and second opening 330 of the vessel 215 are indicated. In the exemplary case, the gasket 360 around the first opening 320 acts as a passive valve controlling flow of gas from the reservoir 610 into the vessel 215 and is discussed with reference to FIG. 10. An electromechanical actuation subsystem 910 is used to control flow of scented gas from the vessel 215 via a channel 920 to the outlet 120 and is discussed with reference to FIG. 11. The cartridge interlayer 350 is indicated adjacent to the vessel 215. The gaskets 370, 380 that surround the second opening 330 and channel 920, respectively, are not visible in the cross-sectional view.

FIG. 10 is an expanded view of aspects of the cross-sectional view shown in FIG. 9. As noted, the exemplary embodiment illustrated in FIGS. 9 and 10 shows the gasket 360 acting as a passive valve at the first opening 320. Until the vessel 215 is filled, gas that is pressurized in the reservoir 610 or directed via a fan or pump from the reservoir 610, for example, is allowed to flow from the reservoir 610 into the vessel 215. The gasket 360 may act as a check valve to prevent the flow of scented gas out of the first opening 320 and back into the reservoir 610. The gasket 360 acting as a check valve can allow the reservoir 610 to be hyper-pressurized. Any vessel 215 that emits scented gas via the second opening 330 may immediately be re-filled with gas, preventing a delay in any requested scent emission. In this exemplary case, a sheet 302 (e.g., film, laminate) on the cartridge 210, between the cartridge 210 and cartridge interlayer 350, may have openings that align with the first openings 320 of the vessels 215 to permit the flow of gas from the reservoir 610 into the vessels 215 via the first openings 320.

FIG. 11 is an expanded view of aspects of the cross-sectional view shown in FIG. 9. As noted, an electromechanical actuation subsystem 910 is used at the second opening 330. According to an exemplary embodiment, the electromechanical actuation subsystem 910 may include the sheet 302 acting as a valve and a thermoelectric actuator. For example, the sheet 320, which does not include holes aligned with the second openings 330, may be a normally closed valve that prevents the out-flow of scented gas from the vessels. The exemplary electromechanical actuation subsystem 910 may include a stopper 1110 that keeps the sheet 302 pressed against the second openings 320. For example, the stopper 1110 may be in the form of a ceramic ball, as shown in FIG. 11, positioned at the second opening 330.

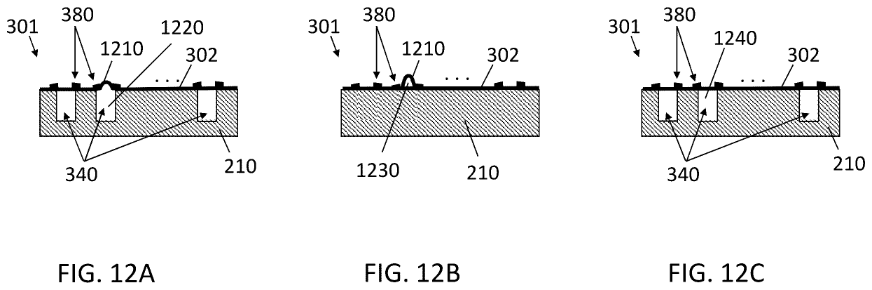

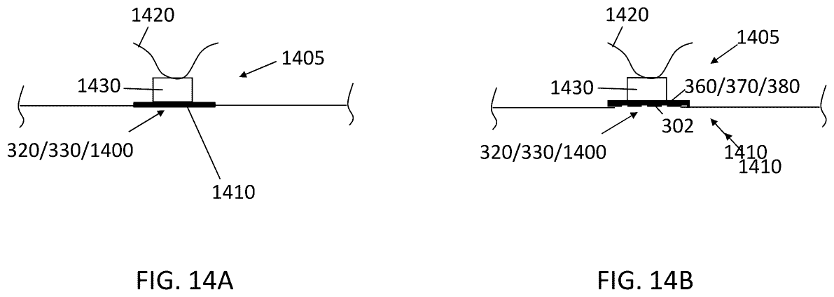

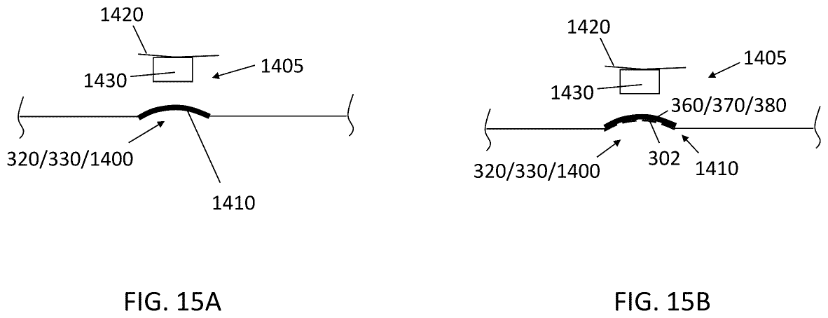

The actuation subsystem 910 may also include a shape memory alloy 1120 such as a nickel-titanium alloy (Nitinol), further discussed with reference to FIGS. 14A, 14B, 15A and 15B, that may be positioned to displace the stopper 1110 from the second opening 330. That is, the shape memory alloy 1120 may contract when a current is applied via the controller 510. Based on the positioning of the shape memory alloy 1120, contraction may result in the stopper moving away from the second opening 330 in the direction indicated by the arrow. This may allow gas that entered the vessel 215 via the first opening 320, which has become scented gas in the vessel 215 due to the scented medium 310, to flow out of the second opening 330, through a channel 920, and out of the device 100 via the outlet 120. Specifically, as discussed with reference to FIG. 3B and further detailed in FIG. 11, the sheet 302 may balloon out away from the second opening 330 based on out-flow of the scented gas from the vessel 215 according to some embodiments, as discussed with reference to FIG. 12A, for example. The flow of scented gas may be via a channel 920 defined by the sheet 302 and a groove 340 in the cartridge 210, for example. This and other channel configurations are discussed with reference to FIGS. 12A, 12B, and 12C.

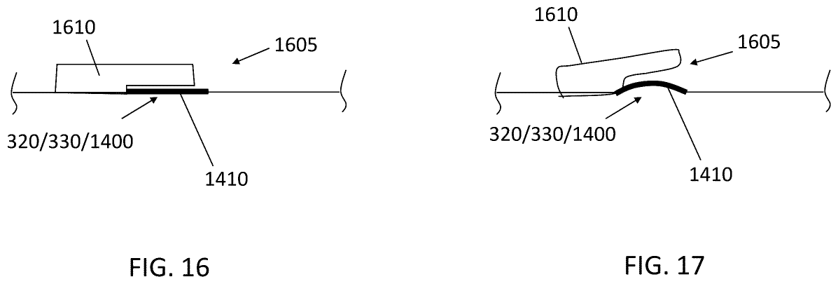

In alternate embodiments, electrostatic effect (e.g., fluid shape control) or electrodynamic effect (e.g., ion generation) may be used for mechanical actuation of the first openings 320 and/or the second openings 330. These are further discussed with reference to FIGS. 16-19. As previously noted, generally one of the openings (first openings 320 or second openings 330) may be controlled via an electromechanical actuation subsystem. The other openings (second openings 330 or first openings 320) may have passive valves (e.g., in the form of gaskets 370, 360) or electromechanical actuation subsystems to control flow.

FIGS. 12A, 12B, and 12C illustrate a cross-section along the length from the second openings 330 to the outlets 120. FIG. 12A is a view from the perspective of the outlets 120, similar to the view shown in FIG. 3B. The grooves 340 in the cartridge 210 are shown and the side 301 of the cartridge that couples to the device 100 is indicated. A sheet 302 covering an open side of the grooves 340 is shown during delivery of scented gas from one of the vessels 215 (second vessel 215 from the left in the example shown). A gasket 380 is on each side of the portion of the sheet 302 that covers each groove 340, as the illustrations in FIG. 3B indicate.

As indicated in FIG. 12A, when scented gas exits the vessel (second vessel 215 from the left in the example shown), the sheet 302 covering the associated groove 304 js pushed away based on pressure of the exiting scented gas, as indicated by the bump 1210. The bump 1210 is contained to the area of the sheet 302 over the associated groove 340 by the gasket 380 on either side of the bump 1210. The bump 1210 and groove 340 form the channel 1220 (e.g., similar to channel 920 in FIGS. 9 and 11). The sheet 302 covering the associated groove 340 and the associated second opening 330 of the vessel 215 from which the scented gas is output may be pushed away based on an actuation subsystem (e.g., 910) controlling flow from the second opening 330. That is, the scented gas is held in the vessel 215 until the second opening 330 is unblocked, at which time the pressure of outflowing scented gas may push the sheet 302 to form the bump 1210 and flow out via the channel 1220. The second opening 330 may be unblocked based on actuator elements of an actuation subsystem (e.g., 910, 1405, 1605, 1805 (FIGS. 11 and 14A-19)).

FIG. 12B is a block diagram that shows a channel 1230 according to an alternate embodiment. In the exemplary embodiment shown in FIG. 12B, the cartridge 210 does not include grooves 340. In this case, the scented gas exiting a given vessel 215 pushes the portion of the sheet 302 between its second opening 330 and the outlet 120 away from the surface of the cartridge 210 to form a channel 1230. As indicated, gaskets 380 may facilitate localizing the disengagement of the sheet 302 from the surface of the cartridge 210.

FIG. 12C is a block diagram that shows a channel 1240 according to another alternate embodiment. In the exemplary embodiment shown in FIG. 12C, the cartridge 210 includes grooves 340, but the sheet 302 may not be compliant enough to be pushed away, based on the pressure of exiting scented gas, to form a bump 1210, as shown for the embodiments of FIGS. 12A and 12B.

In addition, a given path from a second opening 330 to an outlet 120 may include a combination of two or more channels 1220, 1230, 1240. For example, FIG. 3B, which shows the side 301 of the cartridge 210 that couples to the device 100, shows that the exemplary cartridge 210 has grooves 340 that do not begin at the second openings 330. Thus, between a given second opening 330 and corresponding groove 340, a channel 1230 may be formed, and from the start of the corresponding groove to the corresponding outlet 120, a channel 1210 (or 1240) may be formed.

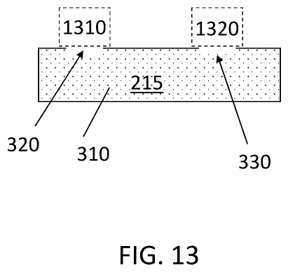

FIG. 13 is a block diagram of a generalized exemplary vessel 215 of a digital scent delivery device 100 according to some embodiments. The vessel 215 is shown with a scent medium 310 in the vessel 215. The scent medium 310 may be, for example, a foam, wax, paper substrate, liquid, emulsion, gel, cotton ball, cotton pad. Gas that enters the vessel 215 becomes scented gas. The first opening 320 for entry of gas (e.g., air, liquified gas) into the vessel 215 is indicated with a first valve 1310 controlling the flow into the first opening 320. The first valve 1310 may be a passive valve formed by a sheet 302 and/or gasket 360, for example. In alternate embodiments, the first valve 1310 may be part of an actuation subsystem according to any of the embodiments discussed herein. In this case, the first valve 1310 may be closed by default and opened based on control of an actuator. For example, as shown in FIG. 9, an exemplary actuation subsystem 910 involves a sheet 302 acting as a valve and an electromechanical actuator employing thermoelectric, electrostatic effect, or electrodynamic effect to control opening of the first opening 320. The first valve 1310 prevents scented gas in the vessel 215 from exiting via the first opening 320. This ensures that the reservoir 610 is not contaminated with scent and does not subsequently cross-contaminate the scent in another vessel 215, for example.

The second opening 330 for exit of scented gas from the vessel 215 is indicated with a second valve 1320 controlling the flow out of the second opening 330. As discussed with reference to the first valve 1310, the second valve 1320 may be a passive valve or may be part of an actuation subsystem. One or both of the first valve 1310 or the second valve 1320 may be part of an actuation subsystem. When both the first valve 1310 and the second valve 1320 are part of an actuation subsystem, they may be part of the same or a different type of actuation subsystem (e.g., thermoelectric, electrostatic, electrodynamic).

FIGS. 14A, 14B, 15A and 15B are used to illustrate closed and open states of the first opening 320, second opening 330, or outlet path 1400 based on an exemplary actuation subsystem 1405 employing thermoelectric effect. Although the system may employ thermoelectric effect, it should be appreciated that in some embodiments, any electromechanical elements may be used including piezoelectric devices or other type of element. The outlet path 1400 refers to any part of the path between a second opening 330 and a corresponding outlet 120. The actuation subsystem 1405 may include a valve 1410. The valve 1410 may be the sheet 302, gasket 360 (if covering the first opening 320), gasket 370 (if covering the second opening 330), gasket 380 (if covering the outlet path 1400) or other component that blocks flow of gas into the vessel 215, flow of scented gas out of the vessel 215, or flow of scented gas out of the device 100 as shown in FIG. 14A. For example, in the case of the valve 1410 over the outlet path 1400, the valve 1410 may be the sheet 302 above the surface of the cartridge 210 (as in FIG. 12B) or above a groove 340 in the cartridge 210. In some embodiments, the valve 1410 may be a combination of the sheet 302 and gasket 360, 370, 380, as shown in FIG. 14B. As noted with reference to FIG. 3B, the sheet 302 may be a film. Thus, the exemplary embodiment shown in FIG. 14B may involve a film and gasket 360, 370, or 380 covering the first opening 320, second opening 330, or outlet path 1400.

The actuation subsystem 1405 may also include actuation elements. For example, a thermoelectric actuator may include a shape memory alloy 1420 (e.g., nitinol) and a stopper 1430 (e.g., ceramic ball stopper 1110) positioned to keep the valve 1410 in the closed position, illustrated in FIGS. 14A and 14B, as a default. Based on application of current to the shape memory alloy 1420 (e.g., via controller 510 operation), the shape memory alloy 1420 may deform, as shown in FIGS. 15A and 15B. As a result, the stopper 1430 may move, as shown, and open the first opening 320, second opening 330, or outlet path 1410 (e.g., channel 1220, 1230, 1240). While the exemplary actuation subsystem 1405 operates based on thermoelectric effect, alternate embodiments may, instead, operate based on electrostatic, electrodynamic, or reverse piezoelectric effect to cause mechanical motion based on an electric control signal. Piezoelectric and electrostatic electromechanical actuation subsystems 1605, 1805 are discussed with reference to FIGS. 16-19. According to exemplary embodiments, as discussed with reference to FIGS. 16-19, the actuator may act directly on the valve 1410 rather than indirectly, by acting on a stopper 1430.

FIGS. 16 and 17 are used to illustrate closed and open states of the first opening 320, second opening 330, or outlet path 1400 based on an exemplary actuation subsystem 1605 employing piezoelectric effect. As noted, another variation shown in FIGS. 16 and 17, as compared with FIGS. 14A, 14B, 15A, and 15B, is the lack of a separate stopper and actuator. That is, FIGS. 16 and 17 show a piezoelectric element 1610 that blocks a valve 1410 from opening, according to the illustration in FIG. 16, and that deforms based on application of current to allow the valve 1410 to open, as illustrated in FIG. 17. In alternate embodiments, a separate stopper may be used with the piezoelectric element 1610 for operation similar to the embodiment shown in FIGS. 9, 11, 14A, 14B, 15A, and 15B.

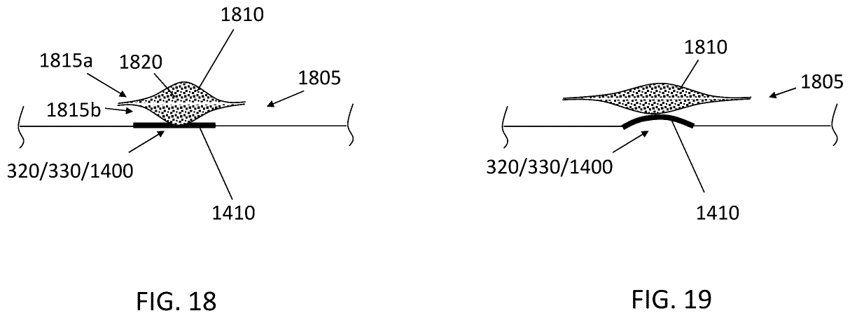

FIGS. 18 and 19 are used to illustrate closed and open states of the first opening 320, second opening 330, or outlet path 1400 based on an exemplary actuation subsystem 1805 employing electrostatic effect. As shown, the actuator 1810, which also acts as a stopper to keep the valve 1410, as illustrated in FIG. 18, includes two surfaces 1815a, 1815b that hold a (dielectric) liquid 1820 therebetween. When current is applied, the surfaces 1815a, 1815b flatten, thereby allowing the valve 1410 to open, as shown in FIG. 19. As noted, in additional alternate embodiments, electrodynamic effect may be used to control a valve 1410.

FIG. 20 is a block diagram of a digital scent delivery device 100 according to some embodiments. The scent delivery device 100 in FIG. 20 is a generalized version of the scent delivery device 100 shown according to exemplary embodiments in prior figures. A controller 10 may communicate with an external device 15 to select one or more scents for delivery. Each scent may be stored in a scent medium 310 in a vessel 30. Each vessel 30 may include a first opening valve 25 (e.g., first opening 320 and passive valve or actuation subsystem) that controls input of a gas (e.g., air, liquified gas) into the vessel 30 from a corresponding reservoir 20.

In the exemplary digital scent delivery device 100 shown in FIG. 20, each vessel 30 is supplied by a corresponding reservoir 20. Each reservoir 20 has an associated gas moving device 22 (e.g., fan, air pump) as the gas supply device 410 according to the exemplary embodiment. The controller 10 may control the gas moving device 22. The controller 10 may also communicate with an EEPROM 40 or other memory that stores information about the scent stored by each vessel 30 and other information (e.g., usage information).

Each vessel 30 includes a second opening valve 35 (e.g., second opening 330 and passive valve or actuation subsystem) that controls output of scented gas from the vessel 30. The digital scent delivery device 100 may include a mixing area 50 according to some embodiments. When a combination of scents is delivered, the scents may be combined in an optional mixing area 50 prior to being released from the digital scent delivery device 100 via one or more outlets 120.

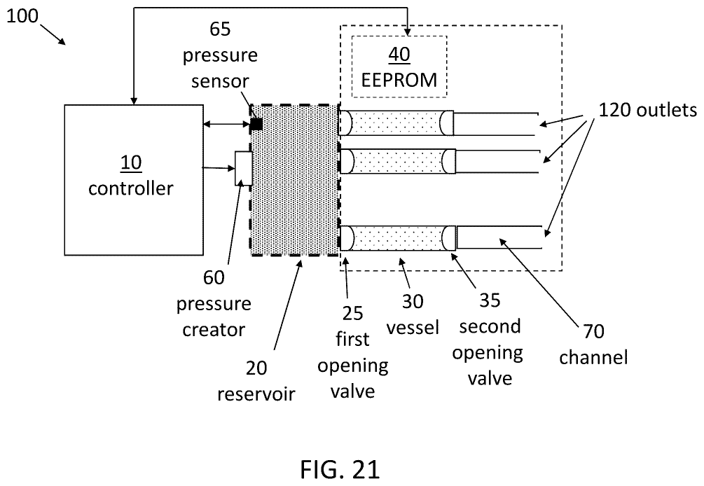

FIG. 21 is a block diagram of a digital scent delivery device 100 according to some embodiments. The scent delivery device 100 in FIG. 21 is a generalized version of the scent delivery device 100 shown according to exemplary embodiments in prior figures and shows some variations from the features shown in FIG. 20. For example, the exemplary embodiment shown in FIG. 21 does not include a mixing area 50. In this type of embodiment, a deflector may be added at one or more outlets 120 to deflect output of the scent medium 310 in one or more of the vessels 30. For example, a deflector (e.g., plastic) may be added at the outlets 120 on the two ends to direct the scent media 310 from those outsets 120 closer to the center. Deflectors may be used at one or more outlets 120 to control directionality of scent for any reason. Additionally or alternately, a fan or other amplification device may be added at one or more outlets 120 to increase the flow velocity from one or more vessels 30 or the mixing area 50. In some embodiments, the controller 10 may modulate intensity of one or more scents by controlling the flow actuation and/or amplification. For example, the EEPROM 325 or other storage associated with the cartridge 210 may specify scent intensity or scent mixing by weight for different combinations of scents. It should be understood that the features shown and discussed with reference to one or more figures may be combined according to contemplated alternate embodiments.

The exemplary embodiment of FIG. 21 shows one reservoir 20 for supply of gas to each of the vessels 30. The reservoir 20 may have a pressure creator 60 (e.g., piezoelectric-based device) as the gas supply device 410. A pressure sensor 65 may monitor pressure in the reservoir 60 and communicate with the controller 10 to facilitate control of the pressure via control of the pressure creator 60 by the controller 10 based on the sensed pressure. In some embodiments, data from the pressure sensor 65 may be used to guide functionality. For example, if the pressure sensor 65 indicates that pressure in the reservoir 60 is below a threshold value, scent delivery may be paused/delayed or another component (e.g., piezoelectric-based device 411, external device) may be signaled to increase pressure in the reservoir 60. The reservoir 20 is shown with dashed lines to indicate that a reservoir 20 as such may not be used at all according to exemplary embodiments. Instead, for example, air may be pulled into the device 100 and supplied (e.g., with or without pressurization) to the vessels 30.

It should be appreciated that there are other applications of this technology and the invention is not limited to the examples provided herein. The device may be incorporated in part or whole into other devices or connected to other devices. For example, some embodiments may be used in general entertainment, which could be movies or other experiences. Additionally, some embodiments may be applied to areas such as travel, business, education/training, telepresence, and meditation.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A system comprising:
a plurality of vessels, each of the plurality of vessels including a scented medium between a first opening and a second opening;
a first valve disposed at the first opening of each of the plurality of vessels;
a second valve disposed at the second opening of each of the plurality of vessels, wherein for each of the plurality of vessels, at least one of the first valve or the second valve is part of a first electromechanical actuation subsystem and another of the first valve or the second valve is a passive valve or is part of a second electromechanical actuation subsystem;
one or more reservoirs holding a gas;
one or more interconnections arranged to supply the gas from the one or more reservoirs to the first opening of the plurality of vessels;
a controller configured to control release of scented gas from at least one of the plurality of vessels via the second opening of the at least one of the plurality of vessels to a corresponding outlet.

2. The system of claim 1, wherein the scented gas results from the gas from one of the one or more reservoirs interacting with the scented medium in the at least one of the plurality of vessels.

3. The system of claim 1, wherein the scented medium is foam, wax, a paper substrate, gel, cotton ball, cotton pad, or porous polymer.

4. The system of claim 1, wherein the first electromechanical actuation subsystem and the second electromechanical actuation subsystem are a same type of electromechanical actuation subsystem.

5. The system of claim 1, wherein at least one of the first electromechanical actuation subsystem or the second electromechanical subsystem includes a thermoelectric actuator.

6. The system according to claim 5, wherein the thermoelectric actuator includes a shape memory alloy configured to move based on an application of a current and a stopper configured to cover at least one of the first opening or the second opening until movement of the shape memory alloy moves the stopper.

7. The system of claim 5, wherein the thermoelectric actuator includes Nitinol.

8. The system of claim 1, wherein at least one of the first electromechanical actuation subsystem or the second electromechanical actuation subsystem operates based on an electrostatic or electrodynamic effect.

9. The system of claim 1, wherein at least one of the first electromechanical actuation subsystem or the second electromechanical actuation subsystem includes a piezoelectric actuator.

10. The system of claim 1, wherein the gas is pressurized air.

11. The system of claim 1, wherein the gas is liquified gas.

12. The system of claim 11, wherein the liquified gas is carbon dioxide.

13. The system of claim 1, further comprising a fan or air pump coupled to the one or more reservoirs to urge flow of the gas from the one or more reservoirs.

14. The system of claim 1, wherein the controller is configured to communicate with an external device and control release of the scented gas based on communication from the external device.

15. The system of claim 14, wherein the controller is configured to communicate with the external device wirelessly or via a port.

16. The system of claim 1, further comprising a cartridge configured to house the plurality of vessels.

17. The system of claim 16, further comprising a cartridge interface, wherein the cartridge is configured to detach from the system and the cartridge interface is configured to couple to the cartridge when the cartridge is attached to the system.

18. The system of claim 17, wherein the cartridge interface includes first openings configured to align, respectively, to the first openings of the plurality of vessels housed in the cartridge, and first gaskets configured to seal an interface between the first openings of the cartridge interface and the first openings of the plurality of vessels, respectively.

19. The system of claim 17, wherein the cartridge includes grooves defining each channel from the second opening of each of the plurality of vessels to each corresponding outlet and the cartridge interface includes channel gaskets configured to seal each of the channels.

20. The system of claim 17, wherein the cartridge includes a sheet covering a side of the cartridge that couples to the cartridge interface.

21. The system of claim 20, wherein each channel is between the sheet and one of the grooves.

22. The system of claim 19, wherein the sheet is a film or a laminate.

23. The system of claim 16, further comprising a removable cover configured to cover the cartridge.

24. The system of claim 16, wherein the controller obtains information from the cartridge.

25. The system of claim 24, wherein the information identifies a scent stored in each of the plurality of vessels.

26. The system of claim 24, wherein the information is stored in an electrically erasable programmable read-only memory (EEPROM) of the cartridge.

27. The system of claim 24, wherein the information indicates allowed and disallowed combinations of scents.

28. The system of claim 24, wherein the information indicates usage of one or more of the plurality of vessels.

29. The system of claim 24, wherein the information is used to replace the cartridge.

30. The system of claim 24, wherein the information is used to refill or replace one or more of the plurality of vessels.
